(19) United States
(12) Patent Application Publication
(10) Pub. No.: US 2010/0203574 A1
(43) Pub. Date: Aug. 12, 2010

(54) METHOD AND APPARATUS FOR CULTIVATING LIVING CELLS

(75) Inventors: Volker Burkart, Duesseldorf (DE); Thomas Burkart, Duesseldorf (DE); Andreas Burkart, Duesseldorf (DE)

Correspondence Address: JOYCE VON NATZMER;PEQUIGNOT + MYERS LLC, 200 Madison Avenue, Suite 1901, New York, NY, 10016, US

(73) Assignee: DEUTSCHE DIABETES-FOSCHUNGSGESELLSCHAFT E.V., Duesseldorf (DE)

(21) Appl. No.: 12/671,658

(22) PCT Filed: Jul. 24, 2008
(86) PCT No.: PCT/DE08/01206

§ 371 Date: Mar. 25, 2010

(30) Foreign Application Priority Data
Aug. 2, 2007 (DE) 10 2007 036 611.8

(51) Int. Cl. C12Q 1/02; C12N 5/071; C12M 3/00

(52) U.S. Cl. 435/29; 435/325; 435/303.1

(57) ABSTRACT

The invention relates to a method for cultivating living cells 6 in a liquid 5, wherein the cells 6 are introduced into at least one culture cavity 3, which is connected with at least one reservoir 2 for the liquid 5. The invention further relates to an apparatus 10 for cultivating living cells 6 in a liquid 5, which comprises at least one culture cavity 3, which is, via at least one connecting element 11, connected with at least one reservoir 2 to hold the liquid 5. In that, according to the invention, it is provided that the volume of the liquid 5 is kept constant in at least one culture cavity 3, preferably by a valve-like mechanism arranged in the connecting element 11.

### FIELD OF THE INVENTION

The invention relates to a method for cultivating living cells in a liquid, wherein the cells are introduced into at least one culture cavity which is in connection with at least one reservoir for the liquid. The invention further relates to an apparatus for cultivating living cells in a liquid, which comprises at least one culture cavity which is in connection with at least one reservoir to receive the liquid via at least one connecting element. The invention also relates to uses of this appliance.

### BACKGROUND OF THE INVENTION

Isolated cells, cell populations or organs up to intact individuals like mice and humans represent complex biological systems, the individual components of which communicate with one another in a varied manner. This communication substantially takes place via so-called “mediators”, which can also be designated as “messenger substances” or “soluble messenger substances”. These messenger substances include, for example, hormones or certain groups of proteins, like cytokines or chemokines, which are primarily formed by immunocytes. In the broader sense, immunoglobulins (antibodies) could also be included in this group of substances. Messenger substances released by cells or tissues have a certain “long-range effect”, i.e. they not only have an effect in the immediate proximity of the cell, from which they are released, but they may also systemically influence entire organs or the entire body. With this “long-range effect”, the messenger substances thus contribute, for example, to the controlled sequence of immunoreactions, or they regulate the response of cells or organ systems to changes in the metabolic status. Since the messenger substances are relatively stable (long-lived), they can accumulate in cell culture supernatants (or also in the serum of animals, patients), where they then may also be quantified with respective specific and sensitive verification procedures. With the impact of pharmacological substances, during the course of inflammations or in certain metabolic situations, the release of these messenger substances can be decisively influenced.

Increasingly, the isolation of small and smallest, partially highly specialized cell populations from animal and human sample material, for example populations of immunocytes and endocrine cells, and their use for research purposes are gaining higher significance. Long-term observation of the functions of these sensitive primary cell populations in vitro frequently requires multiple extractions of aliquots of the culture supernatant for the determination of released mediators. In that, long-term culture under various experimental conditions makes high demands on the cultivation methods, which are to be as sensitive as possible. Currently, experiments are prepared with low cell counts, predominantly in Petri dishes with a volume of 2 to 5 ml or in the cavities of multi-well plates, for example ½ area 96-well micro-titer plates with a volume of 100 μl.

Cultivation in Petri dishes, however, has the disadvantage, that there is high demand for substances, which have to be used for modulation of the cell functions, which results in very high costs. Furthermore, the mediators released by the cells are highly diluted, so that verification of the mediators in the culture supernatant is either not possible or only with difficulty. Cultivation in Petri dishes has the further disadvantage, that very high fluctuations between the preparations in various Petri dishes within one preparation may occur, which has a strong negative impact on the reproducibility of the results. Furthermore, missing or low cell-cell contacts result in a corruption of the cell reactions.

However, the cultivation in micro-titer plates, too, is associated with considerable disadvantages. Accordingly, repeated extraction of aliquots of the culture medium from the wells for the measurement of released mediators respectively requires the addition of fresh medium for volume compensation. Thus, for example, due to the addition of pipetting errors over the duration of the test, high differences in volume could result between the individual cavities. Furthermore, the medium remaining over the cells in the cavities and the medium aliquots added for volume compensation may differ considerably in temperature, pH value, p0₂, etc. Thus, with each new addition of medium, there is an irritation of the cultivated cells, which disturbs the continuity of the culture conditions.

From U.S. Pat. No. 5,578,490, for example, a cell culture plate is known, in which the actual culture cavities are respectively separated from a reservoir allocated to the respective culture cavity by a membrane. The membrane is permeable for substances dissolved in the cell culture medium, but retains the cells in the culture cavity. Thus, dissolved substances, like for example nutrients, may diffuse from the reservoir into the culture cavity, as may dissolved substances, like for example metabolites, from the culture cavity into the reservoir. This cell culture plate therefore has the disadvantage, that mediators to be determined also diffuse out of the culture cavity, so that the concentration of the mediators released by the cells is decreased considerably. Consequently, here, verification of mediators in the culture supernatant is not possible or only with difficulties.

Furthermore, from U.S. Pat. No. 5,726,060, a method for cultivating respiratory epithelial cells of mammals is known, in which the cells are cultivated in an insert having a base permeable for liquids, on which the cells can grow adherently. The insert is placed into a culture dish, which serves as the reservoir for the culture medium. By exchanging the medium in the culture dish, the cells can thus be provided with fresh medium, without them being damaged or irritated in doing so. However, this method, too, has the disadvantage, that the mediators to be determined can diffuse out of the insert, so that the concentration of the mediators released by the cells is decreased considerably, and thus verification of mediators in the culture supernatant is not possible or only with difficulty.

### SUMMARY OF THE INVENTION

It is the object of the invention to avoid the disadvantages stated and to provide a method and an apparatus of the aforementioned type, with which also small tissue samples and cells can be cultivated in low cell counts over a longer period of time.

According to the invention, the object is solved by a method of the aforementioned type, in which the volume of the liquid in at least one culture cavity is kept constant. Thus, according to the invention, no liquid or medium, respectively, must be refilled or exchanged in the culture cavity, since sufficient liquid can always flow into the culture cavity from the reservoir. With the uniform liquid supply to all culture cavities (replicates) from at least one reservoir, optimal conditions for long-term culture are created. In the method according to the invention, cells and/or tissues are for example incubated in a culture cavity with culture medium or similar nutrient fluids, preferably in a small volume (approx. 100 μl). The culture cavity is connected with a reservoir, which preferably contains a larger volume of the culture medium (>5 ml), so that a continuous passover of liquid from the reservoir into the culture cavity is made possible allowing for continuous volume compensation, wherein uniform volume compensation takes place in all culture cavities of a test set-up. Keeping the volume in the culture cavity constant, culturing of small tissue samples or low cell counts in a small volume becomes also possible over a longer period of time, which again results in an improvement of the cell-cell contacts, higher accumulation of released mediators and a lower demand for substances for modulation of the cell/tissue functions. Thus, using the method according to the invention, the release of mediators or messenger substances, respectively, can be exactly examined in a time-dependent fashion under the influences of various experimental conditions (e.g. use of pharmacological substances).

A particularly advantageous embodiment of the method according to the invention provides for the prevention of a pass-over of liquid and/or substances dissolved or suspended in the liquid from the culture cavity into the reservoir, whereby a loss of mediators and thus a reduction of the concentration of these messenger substances can be avoided. In total, a dilution or corruption of the concentrations of the mediators released by the cells is prevented herewith in an advantageous manner, which clearly facilitates the later analysis or only makes it possible at all.

A further advantageous embodiment of the method according to the invention provides for the prevention of the pass-over of liquid and/or substances dissolved or suspended in the liquid from the culture cavity into the reservoir by a valve-like mechanism. On the one hand, this mechanism enables a pass-over of liquid or culture medium, respectively, from the reservoir into the culture cavity for volume compensation, however, on the other hand, it prevents a passover of medium (and modulators, mediators, etc. dissolved therein) from the culture cavity into the reservoir in a very effective manner.

Preferably, within the valve-like mechanism, at least one closing body closes an opening and/or a channel due to its own weight, so that no liquid can escape from the culture cavity into the reservoir. Simultaneously, the closing body, however, allows volume compensation in the culture cavity, when liquid is extracted from it or withdrawn due to evaporation, since due to a reduction of the pressure by the liquid column in the culture cavity, liquid can flow from the reservoir into the culture cavity.

It is particularly advantageous, when the reservoir is arranged in the immediate proximity of the culture cavity, since in this case, volume compensation in the culture cavity can take place with liquid from the adjacent reservoir with an adapted pH value, equal temperature, adapted partial oxygen pressure (p0₂), etc. Furthermore, from the direct vicinity of the reservoir results an improved evaporation protection in the culture cavity. In standard micro-titer/multi-well plates, the evaporation losses are highest in the marginal wells (despite the lid placed on top). Due to the particularly advantageous proximity of the reservoir to the culture cavities, in case of several culture cavities preferably located in the central area of the apparatus according to the invention, the culture cavities, according to the invention, are preferably completely surrounded by the reservoir filled with liquid. The liquid surface of the reservoir, which is very big in comparison to that of the culture cavities, thus keeps the evaporation losses of the centrally located culture cavities extremely low for embodiments with several culture cavities.

In particular for the cultivation of cells, which are available in very low quantities only, it is advantageous, when the culture cavity is filled with a low volume of liquid, preferably a maximum of 200 μl, particularly preferred a maximum of 100 μl or 50 μl.

A particularly advantageous embodiment of the method according to the invention further provides for the reservoir being filled with a volume, which is at least the 5-fold, preferably the 10-fold, particularly preferred the 100-fold of the volume of the liquid in the culture cavity. Due to the fact that the volume in the reservoir is significantly higher than that in a culture cavity or the sum of all culture cavities, continuous volume compensation is guaranteed, without the volume in the culture cavity or in the culture cavities being notably reduced.

According to the invention, the object is further solved by an apparatus of the aforementioned type, in which the connecting element is designed such that the volume of the liquid is kept constant in at least one culture cavity. Due to such a design of the connecting element, a uniform liquid supply into the culture cavity or the culture cavities (replicates), respectively, from a reservoir is possible, in order to create optimal conditions for long-term culture. The connecting element thus enables in an advantageous manner a pass-over of liquid from the reservoir into at least one culture cavity for volume compensation. Keeping the volume in the culture cavity constant, the culture of small tissue samples or low cell counts in a small volume becomes possible also over a longer period of time, which again results in an improvement of the cell-cell contacts, an avoidance of dilution or corruption of the concentrations of the mediators released by the cells and a lower demand for substances for modulation of the cell/tissue functions.

A particularly advantageous embodiment of the apparatus according to the invention provides a connecting element, which comprises at least one opening and/or at least one channel, so that only a limited, well controllable area is available for the pass-over of the liquid.

An advantageous embodiment of the apparatus according to the invention further provides a connecting element, which comprises a valve-like mechanism. The valve-like mechanism enables, in an advantageous manner, a pass-over of liquid from the reservoir into at least one culture cavity. Simultaneously, however, the mechanism also prevents a pass-over of liquid and above all modulators, mediators and the like dissolved therein, from the culture cavity into the reservoir. Consequently, potential target molecules accumulate for later analysis, which especially in case of low cell counts is of particular advantage. A further advantage of the valve-like mechanism according to the invention compared to the known solutions with a membrane lies in the “valve solution” also allowing the pass-over of highly molecular substances from the reservoir into the respective culture cavities. Membranes, on the other hand, usually have a “molecular exclusion limit”, which prevents the pass-over of highly molecular substances, like for example serum proteins. Serum proteins are important components of culture media and absolutely required for the cultivation of most mammal cells. When using membranes, these important medium components may thus not completely pass over from the reservoir into the culture cavity or only in a delayed fashion, thereby damaging the cultured cells. Furthermore, due to charges at the surface of the membrane material (cationic or anionic materials), charged components of the culture medium (e.g. certain amino acids) can be prevented from passing through the membrane. “Valves” of cell culture-suitable plastics, the surfaces of which normally are not charged, do not have these negative characteristics.

A further advantageous embodiment of the apparatus according to the invention provides a valve-like mechanism, which comprises at least one closing body. This closing body can, for example, take the form of a ball, cylinder, cone or valve.

In an advantageous embodiment of the invention, the closing body may also be a sheet. The sheet may, for example, be fixed in the connecting element by gluing or welding. Alternatively, the sheet may also be clamped into a frame or the like, wherein in this embodiment preferably the frame is fastened in the connecting element. In any case, the sheet is fastened freely movable, so that, depending on the pressure conditions respectively present, it either closes or opens the channel of the connecting element. The sheet may, for example, comprise plastic or an impregnated textile material. In any case, the material has to be selected so that the proper function of the closing body is guaranteed. In that, above all weight, elasticity as well as glueability or weldability of the material play an important role.

A particular embodiment provides a plurality of culture cavities, preferably 8, 16 or 32. The apparatus according to the invention may thus also be designed as a multi-well plate or micro-titer plate, respectively, which in an advantageous manner allows its application in serial tests and high-throughput methods (high-throughput screening). In that, the culture cavities are preferably separated from one another, wherein each individual culture cavity has a separate connecting element, so that the volume compensation in each culture cavity can take place without it being possible that cross contaminations occur.

If the apparatus according to the invention comprises at least partially, for example in the base area, clear plastic, continuous microscopic observation of the cultivated cells/tissues as well as an evaluation of cell-/tissue-specific parameters is possible using colorimetric/photometric methods (multi-channel photometer). In an embodiment of the apparatus according to the invention with several culture cavities in the format of a standard micro-titer/multi-well plate, furthermore centrifugation with special centrifuge rotors (inserts) is possible, which are sold by many manufacturers of laboratory centrifuges, exist in most cell/tissue culture laboratories and are used routinely.

The apparatus according to the invention can be used for long-term cultivation of living cells or tissues in low quantities and/or low volume, in particular in multiple preparations for kinetic studies under various experimental conditions. Thus, the apparatus, just like the method according to the invention, is suitable for cultivation and observation of a plurality of cell types (primary cells, cell lines) from humans and animals (e.g. mouse, rat), which only occur in a low quantity or only can be isolated in low quantities, like for example:

Endocrine cells: cells of the adrenal gland, insulin-producing beta cells from the pancreas;

Immunocyte sub-populations: regulatory T-lymphocytes, natural killer cells;

Endothelial cells of blood vessels;

Precursor cells for different tissues: bone, lung epithelium; or

Nerve cells.

The apparatus according to the invention may further be used for the screening of substances, in particular substances with an influence on the survival and/or function (release of mediators like hormones, immunologic messenger substances (cytokines)) of cells and tissues (toxicity tests, examination of pharmacological inhibitors/stimulators). The culture system particularly facilitates sequence analyses for the determination of the point in time at which, for example, the effect of a certain substance occurs or reaches its optimum.

The invention will now, by way of example, be explained in more detail on the basis of the following figures.

### BRIEF DESCRIPTION OF THE FIGURES

FIG. 1 shows a schematic side view (longitudinal section) of an embodiment of the apparatus according to the invention with a reservoir with a volume of about 5 ml and a culture cavity with a volume of about 100 μl.

FIG. 2 shows a schematic side view (longitudinal section) of a further embodiment of the apparatus according to the invention with a valve-like mechanism within the connecting element.

FIG. 3 shows a perspective view of the valve-like mechanism according to FIG. 2.

FIGS. 4-8 show sections through various embodiments of the connecting element with views from the side, and following rotation by 90°, from the front, a) side view, b) front view.

FIG. 9 shows an embodiment of the apparatus according to the invention with 16 culture cavities.

### DESCRIPTION OF EXEMPLARY AND PREFERRED EMBODIMENTS OF THE INVENTION

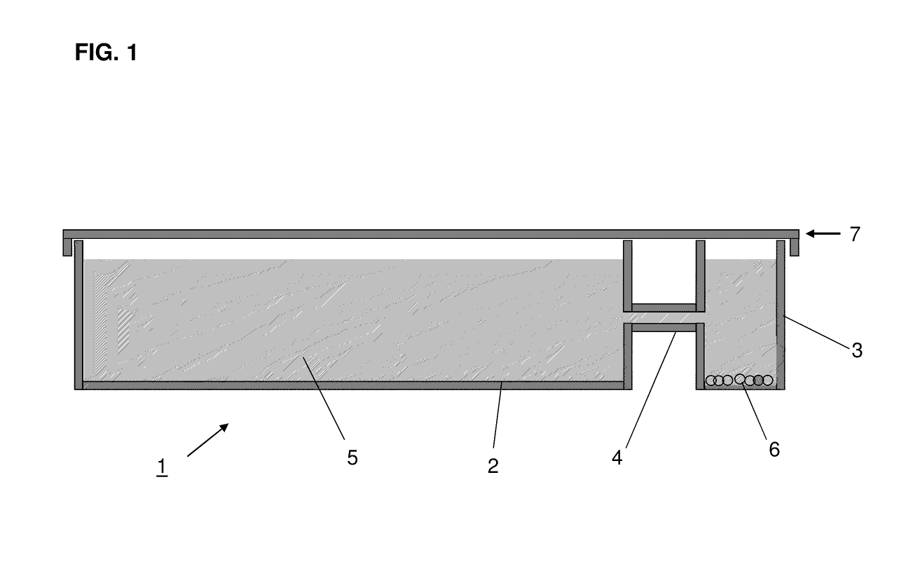

FIG. 1 shows a schematic side view of an embodiment of an apparatus 1 according to the invention with a reservoir 2 and a culture cavity 3. The reservoir 2 is connected with the culture cavity 3 via a connecting element 4. The connecting element 4 has a device or a mechanism not shown here, which prevents that a liquid 5 in the culture cavity 3, which may be, for example, a culture medium for living cells or another nutrient solution, flows from the culture cavity 3 into the reservoir 2. Simultaneously, the device, however, allows a pass-over of the liquid 5 from the reservoir 2 into the culture cavity 3. In this manner, volume compensation in the culture cavity 3 may take place, when liquid 5 is extracted therefrom or, for example, withdrawn therefrom by evaporation. I.e., there automatically and continuously is an adaptation of the height of the liquid level between the reservoir 2 and the culture cavity 3. As soon as the liquid level in the culture cavity 3 declines, liquid 5 flows from the reservoir 2 into the culture cavity 3, until the liquid levels in both compartments have an equal height again. Since in the embodiment shown here the volume of the liquid 5 in the reservoir 2 (5 ml) is clearly higher, i.e. here 50 times higher, than in the culture cavity 3 (100 μl), the volume of the liquid 5 in the culture cavity stays practically constant. Due to the fact that in the embodiment shown here, the reservoir 2 is arranged in the immediate proximity of the culture cavity 3, i.e. these are connected via a short connecting element 4 only, the volume compensation in the culture cavity 3 can take place with the liquid 5 from the adjacent reservoir 2 with an adapted pH value, equal temperature, adapted partial oxygen pressure (p0₂), etc. Hereby, the culture conditions are kept constant, and unnecessary stress for the cells 6 or tissues, respectively, in the culture cavity 3 is avoided. Furthermore, from the direct vicinity of the reservoir 2 results an improved evaporation protection in the culture cavity 3. According to the method of the invention, the apparatus 1 shown here may, for example, be used for the cultivation of living cells 6 in a low quantity and in a small volume. For this purpose, the cells 6 are added to, for example, 50 μl of medium and then introduced into the culture cavity 3. Following sedimentation or centrifuging off of the cells 6, these predominantly are located at the bottom of the culture cavity 3. In an embodiment of the apparatus according to the invention with several culture cavities in the format of a standard micro-titer/multi-well plate, centrifugation with special centrifuge rotors (inserts) is possible as well, which are sold by many manufacturers of laboratory centrifuges, exist in most cell/tissue culture laboratories and are used routinely. Now, a larger volume of medium, for example 5 or 10 ml, can be filled into the reservoir 2, whereupon the liquid level in the culture cavity 3 rises up to the level of the reservoir 2. The cell culture can now be maintained under constant conditions over a longer period of time, wherein the volume in the culture cavity 3 remains approximately constant, even if liquid is extracted for testing purposes.

In this embodiment, the apparatus 1 according to the invention is covered with a removable lid 7 of transparent plastic like for a standard micro-titer/multi-well plate. This lid 7 is primarily important to guarantee the sterility of the system over the duration of the culture. It is not lying flush against the apparatus 1 in a sealing manner, but enables, like the lid of a Petri dish, with little spacers (not shown here), the gas exchange important for the stabilization of the pH value between the culture system and the interior of an incubator.

In standard micro-titer/multi-well plates, the evaporation losses are highest in the marginal wells (despite the lid placed on top). Due to the particularly advantageous proximity of the reservoir to the culture cavities, in case of several culture cavities preferably arranged in the central area of the apparatus according to the invention, the culture cavities are ideally completely surrounded by the reservoir filled with liquid. The liquid surface of the reservoir, which compared to that of the culture cavities is very large, thus keeps the evaporation losses of the centrally located culture cavities extremely low in embodiments with several culture cavities.

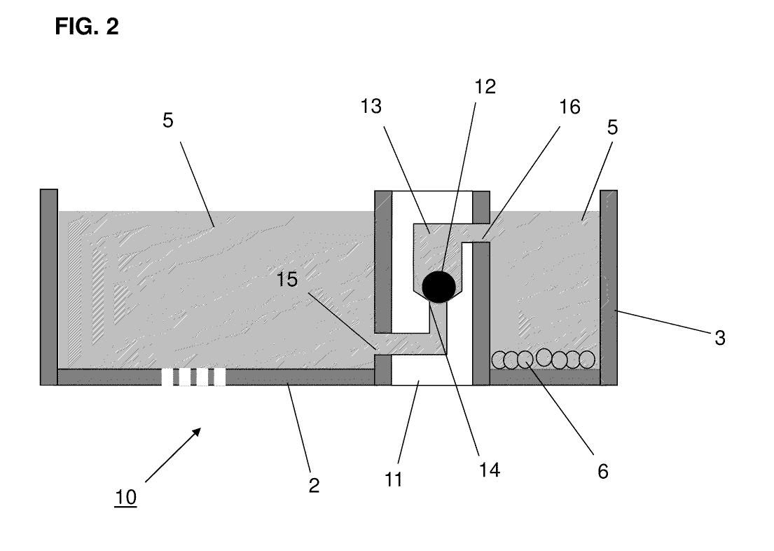

FIG. 2 shows a schematic side view of a further embodiment of an apparatus 10 according to the invention with a valve-like mechanism within a connecting element 11. The valve-like mechanism comprises a cylinder-shaped closing body 12 arranged within the channel 13, which connects the reservoir 2 with the culture cavity 3. The valve-like mechanism in this embodiment is based on the technical principle of “own weight-controlled (check) valves” and enables a directed flow of the liquid 5 from the reservoir 2 into the culture cavity 3.

In that, the valve-like mechanism according to the invention has the following advantages:

high functional reliability,

simple manufacture at the smallest scale possible,

use of cell/tissue culture-compatible plastics possible,

no impairment of the function during transport or storage of the culture systems.

The closing body 12 closes the opening 14 in channel 13 with its own weight, so that no liquid (including the substances dissolved therein) can flow from the culture cavity 3 into the reservoir 2. However, as soon as the pressure of the liquid column in the reservoir 2 becomes higher than the pressure of the liquid column in the culture cavity 3, for example upon extraction of liquid from the culture cavity 3, the closing body 12 is pressed upwards, so that it opens the opening 14. The liquid 5 can then flow from the reservoir 2 into the culture cavity, until the liquid levels in both compartments have adapted again. Then, due to its own weight, the closing body 12 sinks downward again and closes the opening 14, so that no liquid 5 can flow back from the culture cavity 3 into the reservoir 2. The mechanism according to the invention is very effective and guarantees continuous volume compensation in the culture cavity 3, without the substances produced by the cells 6 and to be verified in the culture supernatant being diluted or able to escape. In the present embodiment, the channel 13 of the connecting element 11 is arranged such that the inlet 15, through which the liquid 5 flows from the reservoir 2 into the channel 13, is located in the proximity of the bottom of the reservoir 2, while the outlet 16, through which the liquid 5 flows out of the channel 13 into the culture cavity 3, is located in the upper section of the culture cavity 3. This arrangement has the advantage, that the cells 6 are not irritated by flowing in liquid 5. Thus, in the apparatus 10 according to the invention, the cells 6 can be cultivated in a small volume in an undisturbed manner over a longer period of time.

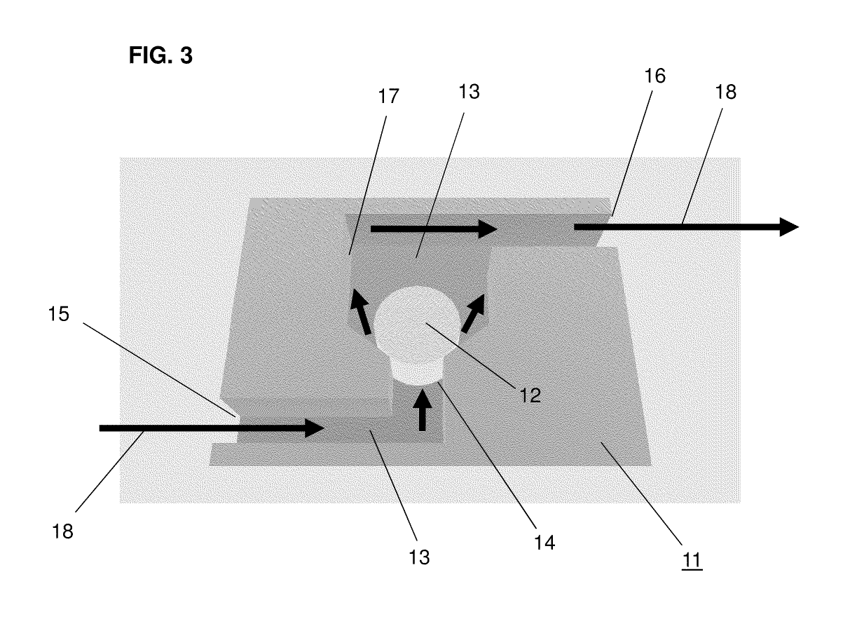

FIG. 3 shows a perspective view of the connecting element 11 of the apparatus 10 according to FIG. 2. Here, it becomes apparent that the closing body 12 has the shape of a flat cylinder and closes the opening 14 by a tight fit to the wall 17 of the channel 13, which is tapered in this area towards the inlet 15. Here, the arrows 18 symbolize the direction of flow of the liquid or the culture medium, respectively, in channel 13.

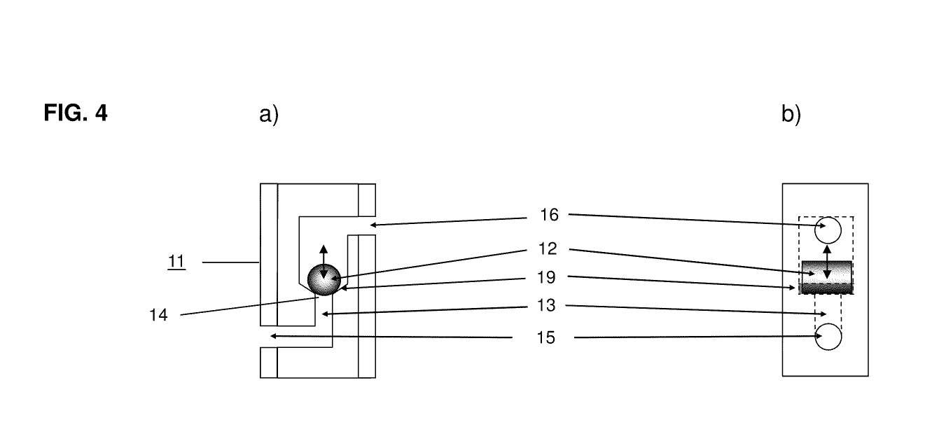

FIGS. 4 to 8 respectively show sections through various alternative embodiments of connecting elements according to the invention in side and front views. For reasons of clarity, the individual components of the connecting elements and the valve-like mechanism are provided with the same reference numbers in FIGS. 4 to 8 like the respective components in FIGS. 2 and 3. In that, the contact surface 19 for the closing body 12 is respectively shown highlighted in gray.

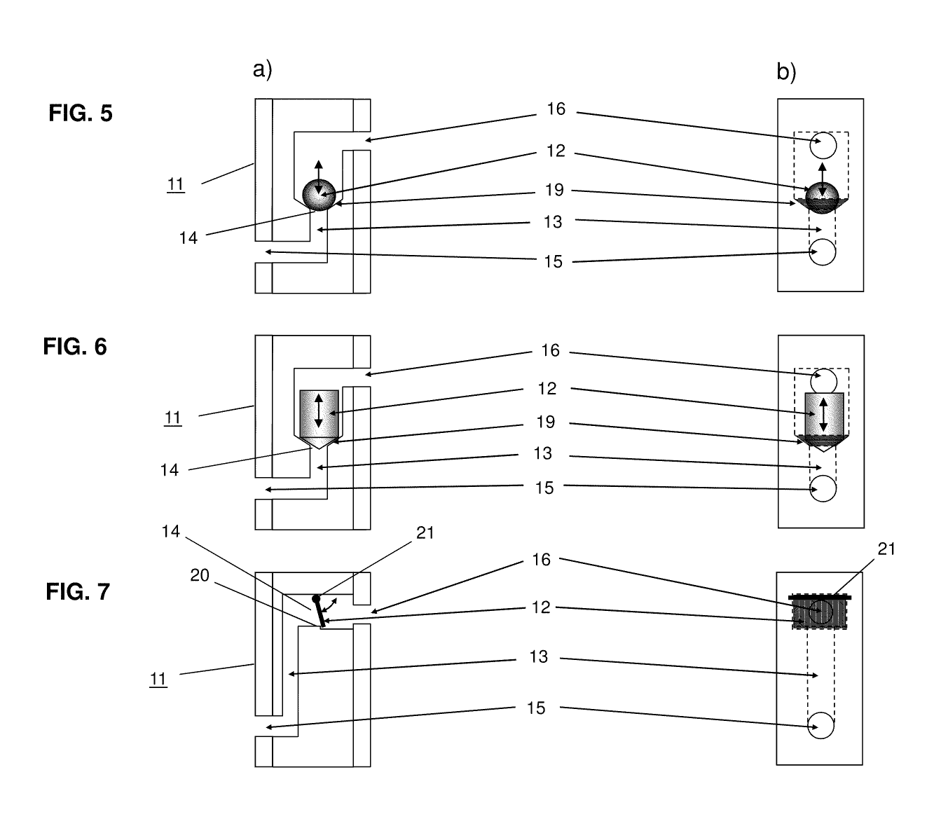

FIG. 4 shows the connecting element 11 with the closing body 12 according to FIGS. 2 and 3. FIG. 5 shows a connecting element 11 with a ball-shaped closing body 12, while FIG. 6 shows a connecting element 11 with a cone-shaped closing body 12. The embodiments shown in FIGS. 2 to 6 have in common, that the channel 13 narrows in the area of the opening 14 towards the inlet 15, so that a contact surface 19 is formed, against which the closing body 12 can lie flush in order to close the opening 14. FIG. 7 shows a further alternative embodiment of the connecting element 11, in which the closing body 12 has the shape of a valve. In this embodiment, the channel 13 has an edge 20 in the proximity of the outlet 16, against which the valve can lie flush, so that it is held in a position, in which the opening 14 is closed. The valve is swiveling towards the outlet 16 around the axis 21.

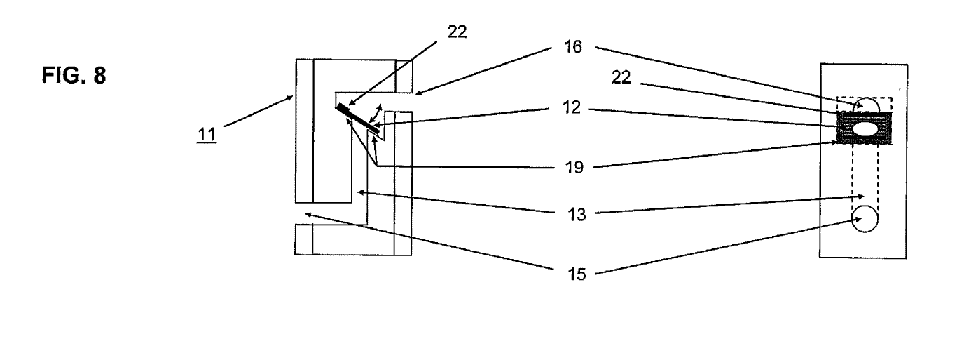

FIG. 8 shows the connecting element 11 with a flat closing body 12, wherein the closing body 12 is a sheet. The sheet is fixed to the connecting element 11 in the fastening area 22, wherein the fastening area 22, for example, may be a gluing or welding seam. Except for the fastening area 22, the sheet is freely movable and thus can be moved in the directions indicated by the double arrow. In its closed condition, the sheet lies flush against the contact surfaces 19 and thus closes the channel 13. In case of excess pressure in the channel 13, however, it can move away from the contact surfaces 19 and thus open the channel 13.

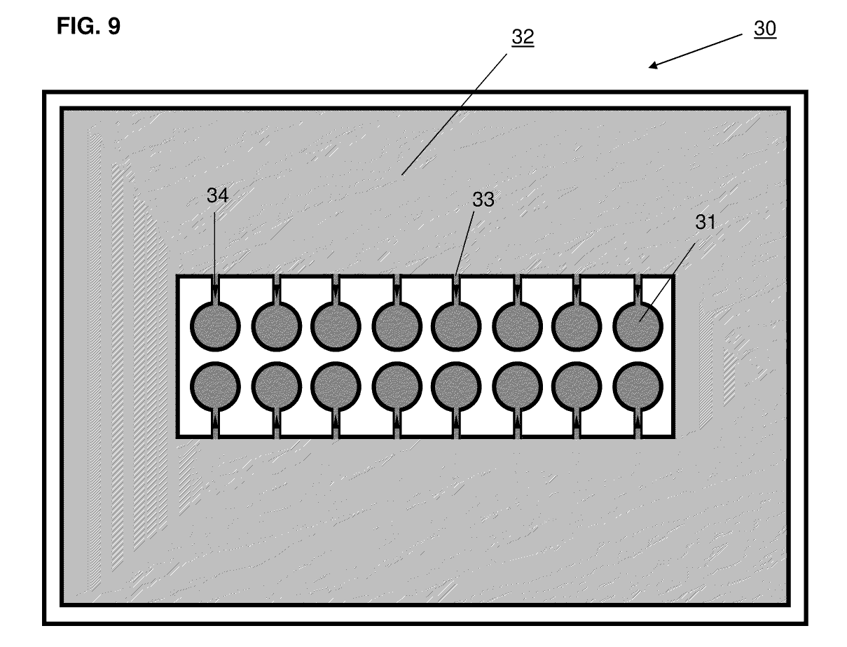

FIG. 9 shows an embodiment of the apparatus 30 according to the invention with 16 culture cavities 31. In this example, the culture cavities 31 are arranged in two parallel rows with eight culture cavities 31 each. Each individual culture cavity 31 is connected with a reservoir 32, which surrounds the arrangement of the culture cavities 31, via a separate connecting element 33. Within the channel-shaped connecting elements 33, valve-like mechanisms are respectively arranged, which are symbolized by the arrows 34. The mode of operation of these valve-like mechanisms corresponds to that as described above in connection with FIGS. 1 to 8 already.

### REFERENCE LIST

1 Apparatus

2 Reservoir

3 Culture cavity

4 Connecting element

5 Liquid

6 Cells

7 Lid

8

9

10 Apparatus

11 Connecting element

12 Closing body

13 Channel

14 Opening

15 Inlet

16 Outlet

17 Wall

18 Arrows

19 Contact surface

20 Edge

21 Axis

22 Fastening area

30 Apparatus

31 Culture cavities

32 Reservoir

33 Connecting element

34 Arrows

1. A method for cultivating living cells in a liquid, wherein said cells are introduced into at least one culture cavity which is in connection with at least one reservoir for said liquid, wherein the volume of said liquid is kept constant in at least one culture cavity.

2. The method according to claim 1, wherein a pass-over of liquid and/or substances dissolved or suspended in said liquid from said culture cavity into said reservoir is prevented.

3. The method according to claim 2, wherein said pass-over of liquid and/or substances dissolved or suspended in said liquid from said culture cavity into said reservoir is prevented by a valve-like mechanism.

4. The method according to claim 3, wherein, within said valve-like mechanism, at least one closing body closes an opening and/or a channel due to its own weight.

5. The method according to claim 1, wherein said reservoir is arranged in the immediate proximity of said culture cavity.

6. The method according to claim 1, wherein said culture cavity is filled with a low volume of liquid, preferably a maximum of 200 μl.

7. The method according to claim 1, wherein said reservoir is filled with a volume, which is at least 5-fold of the volume of said liquid in said culture cavity.

8. An apparatus for cultivating living cells in a liquid, which comprises
at least one culture cavity, which is, via at least one connecting element, connected with at least one reservoir to hold said liquid, wherein said connecting element is designed such that the volume of said liquid is kept constant in at least one culture cavity.

9. The apparatus according to claim 8, wherein said connecting element comprises at least one opening and/or at least one channel.

10. The apparatus according to claim 8, wherein said connecting element comprises a valve-like mechanism.

11. The apparatus according to claim 10, wherein said valve-like mechanism comprises at least one closing body.

12. The apparatus according to claim 11, wherein said closing body has a shape of a ball, cylinder, cone or valve.

13. The apparatus according to claim 11, wherein said closing body is a sheet.

14. The apparatus according to claim 8, wherein a plurality of culture cavities is provided, preferably 8, 16 or 32.

15. The apparatus according to claim 14, wherein said culture cavities are separated from one another, and wherein each individual culture cavity has a separate connecting element.

16. A method for cultivation of living cells, comprising
providing the apparatus of claim 8, and
cultivating said living cells or tissues which are present in low quantities and/or low volume long-term.

17. A method for screening substances, comprising
providing the apparatus of claim 8, and
screening of substances with said apparatus.

18. The method according to claim 6, wherein said culture cavity is filled with a maximum of 100 μl or 50 μl of said liquid.

19. The method according to claim 7, wherein said volume is at least 10-fold of the volume of said liquid in said culture cavity.

20. The method according to claim 7, wherein said volume is at least 100-fold of the volume of said liquid in said culture cavity.
